(19) United States
(12) Patent Application Publication
(10) Pub. No.: US 2024/0423787 A1
(43) Pub. Date: Dec. 26, 2024

(54) Replacement Heart Valve

(71) Applicant: Medical Equipment Design Innovation, Inc., Irvine, CA (US)

(72) Inventor: Pham Cong Lo, Rancho Mission Viejo, CA (US)

(73) Assignee: Medical Equipment Design Innovation, Inc., Irvine, CA (US)

(21) Appl. No.: 18/212,044

(22) Filed: Jun. 20, 2023

(51) Int. Cl. A61F 2/24

(52) U.S. Cl. CPC A61F 2/2418; A61F 2/2433; A61F 2210/0014; A61F 2220/0075

(57) ABSTRACT

A heart valve assembly has a valve frame having a leaflet assembly sutured to the valve frame. The heart valve assembly has an annulus frame having an annulus skirt sutured to the annulus frame. The annulus frame is connected to the valve frame. The valve frame and the annulus frame are made from different materials. A delivery system has a balloon catheter having a shaft, a balloon provided on the shaft adjacent the distal end of the catheter, and a valve frame seat located on the shaft directly proximal to the balloon. The delivery system also has a sheath assembly having a capsule that slidably covers the balloon and the valve frame seat in a manner such that the valve frame is seated around the valve frame seat and the annulus frame is seated over the balloon.

### BACKGROUND OF THE INVENTION

### 1. Field of the Invention

The present invention relates to prosthetic heart valves, and in particular, to a transcatheter aortic heart valve prosthesis that is adapted for treating aortic insufficiency (AI), aortic regurgitation (AR), and aortic stenosis (AS).

### 2. Description of the Prior Art

Transcatheter heart valves are now commonly used to treat valve disease. These heart valves are typically made of a frame that supports a prosthetic heart valve having leaflets that are usually made of bovine pericardium or porcine pericardium. There are a few types of transcatheter aortic heart valves that are available in the market.

One type is manufactured by Medtronic, which has a frame that is made of a shape memory material such as Nitinol. The frame on this heart valve is self-expanding, so this can be used only with patients having AS where the calcification of the native leaflets in the patient is helpful to anchoring this heart valve. However, this heart valve cannot be used for patients with severe calcification. In addition, this heart valve is not optimal for use with patients without calcification in the AI (Aortic insufficiency) or AR (Aortic regurgitation) conditions because the radial force of the frame is low, which means that the frame can easily migrate from its implanted position. Such a heart valve is also difficult to control after it has been expanded and released from the delivery system.

Another type is manufactured by Edwards Lifesciences and uses a frame that is made of cobalt chromium. Since cobalt chromium is a stiffer material and is not a shape memory material, it needs be expanded by a balloon. Balloon expansion also means that it can only be used with patients that have AS where the calcification of the native leaflets of the patient can help to anchor the implanted heart valve. Unfortunately, this heart valve is also not optimal for use patients that do not exhibit calcification at the aortic valve annulus (i.e., in the AI and AR situations) even though the radial force of the frame is strong. This is because the implanted heart valve can still easily migrate. In addition, when the heart valve is crimped on the balloon and during balloon inflation, the folding of the balloon does not provide a precise implantation at the location of the valve annulus because the balloon unfolds in a rolling mechanism.

### SUMMARY OF THE DISCLOSURE

It is an object of the present invention to provide a transcatheter aortic heart valve prosthesis that can be used for treating AI, AR and AS.

It is another object of the present invention to provide a delivery system for delivering the heart valve prosthesis to the aortic annulus in a patient.

To meet the objectives of the present invention, there is provided a heart valve assembly that has a valve frame having a leaflet assembly sutured to the valve frame, the leaflet assembly having a plurality of leaflets. The heart valve assembly has an annulus frame having an annulus skirt sutured to the annulus frame, with the annulus frame connected to the valve frame. The valve frame and the annulus frame are made from different materials. In one embodiment, the valve frame is made from a self-expanding material and the annulus frame is made from a balloon expandable material.

The present invention also provides a delivery system having a balloon catheter having a shaft having a distal end, a balloon provided on the shaft adjacent the distal end, and a valve frame seat located on the shaft directly proximal to the balloon. The delivery system also has a sheath assembly having a capsule that slidably covers the balloon and the valve frame seat in a manner such that the valve frame is seated around the valve frame seat and the annulus frame is seated over the balloon.

### BRIEF DESCRIPTION OF THE DRAWINGS

FIG. 1 is a perspective view of a transcatheter aortic heart valve prosthesis according to one embodiment of the present invention shown with the valve leaflets closed.

FIG. 2 is a perspective view of the heart valve prosthesis of FIG. 1 shown with the valve leaflets open.

FIG. 3 is a side view of the heart valve prosthesis of FIG. 1.

FIG. 4 is a perspective view showing only the valve frame and the annulus frame of the heart valve prosthesis of FIG. 1.

FIG. 5 is a side view of the valve frame and the annulus frame of FIG. 4.

FIG. 6 is a perspective view showing only the valve frame and the leaflet assembly of the heart valve prosthesis of FIG. 1.

FIG. 7 is a perspective view showing only the valve frame of FIG. 6.

FIG. 8 is a side view showing only the valve frame of FIG. 6.

FIG. 9 is a perspective view showing only the leaflet assembly of FIG. 6.

FIG. 10 is a perspective view showing only the annulus frame and the annulus skirt of the heart valve prosthesis of FIG. 1.

FIG. 11 is a perspective view showing only the annulus frame of FIG. 10.

FIG. 12 is a perspective view showing only the annulus skirt of FIG. 10.

FIG. 13 is a schematic view of a delivery system according to the present invention showing the heart valve prosthesis of FIG. 1 compressed on the balloon.

FIG. 14 is a schematic view of the delivery system of FIG. 13 shown without the heart valve prosthesis of FIG. 1, and with the balloon section exposed.

FIG. 15 is a schematic view of the balloon catheter of the delivery system of FIG. 13.

FIG. 16 is a schematic view of the sheath of the delivery system of FIG. 13.

FIG. 17 is a schematic view of the delivery system of FIG. 13 shown with the heart valve prosthesis of FIG. 1 loaded on the balloon, and with the balloon section completely covered by the capsule.

FIG. 18 is a schematic view of the delivery system of FIG. 13 shown with the heart valve prosthesis of FIG. 1 loaded on the balloon, and with the capsule partially withdrawn to partially expose the heart valve prosthesis.

FIGS. 19-21 illustrate the deployment steps of the heart valve prosthesis with the expansion of the balloon and then the withdrawal of the balloon.

FIGS. 22-25 illustrate how the delivery system of FIG. 13 delivers and deploys the heart valve prosthesis of FIG. 1 at the aortic annulus of a human heart.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

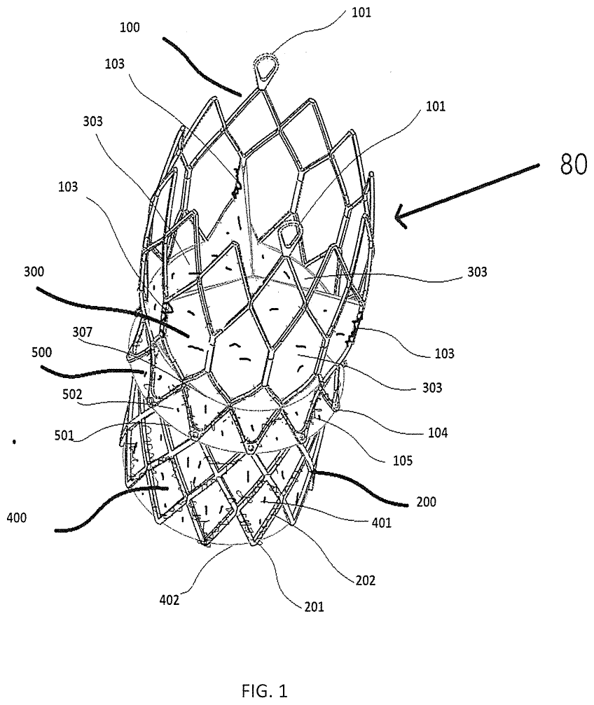

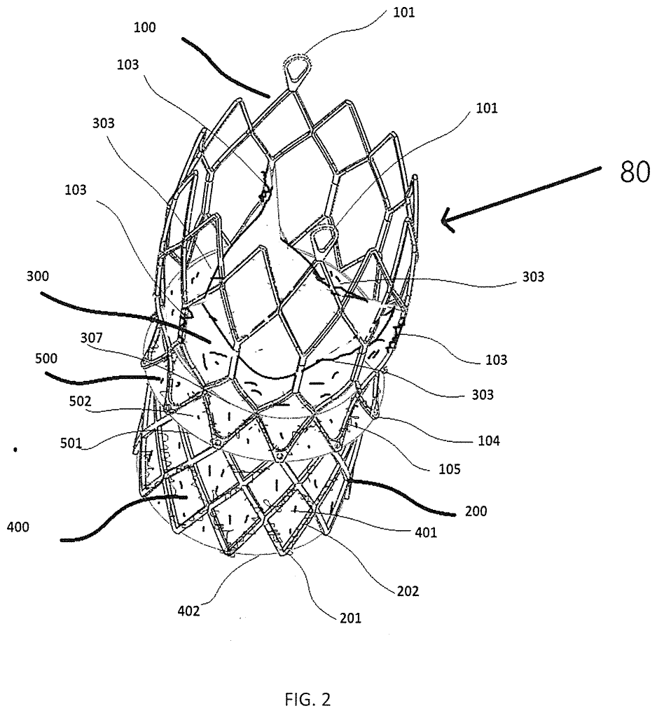

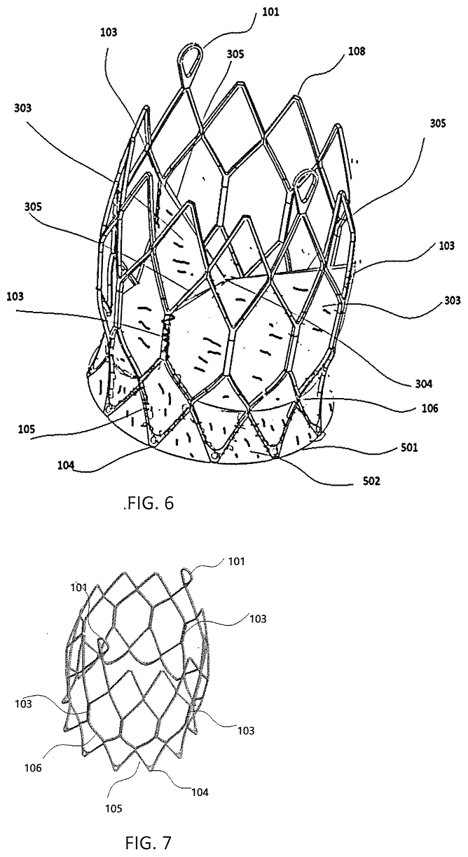

The present invention provides a transcatheter aortic heart valve prosthesis 80 that can be used for treating AI, AR and AS. Referring to FIGS. 1-12, the prosthesis 80 includes a valve frame 100, an annulus frame 200, a tissue leaflet assembly 300 that is retained inside the valve frame 100, and a tissue annulus skirt 400 that is secured to the annulus frame 200. The present invention essentially divides the overall frame of the prosthesis 80 into two separate frames that have two different types of materials that are adapted to provide more effective performance for their intended functions. Specifically, the annulus frame 200 is provided in a more rigid material, such as cobalt chromium, so that it can better perform its intended function of securing the prosthesis 80 to the native annulus, while the valve frame 100 is provided in a more flexible material, such as Nitinol™, so that it can better perform its intended function of supporting the leaflet assembly 300.

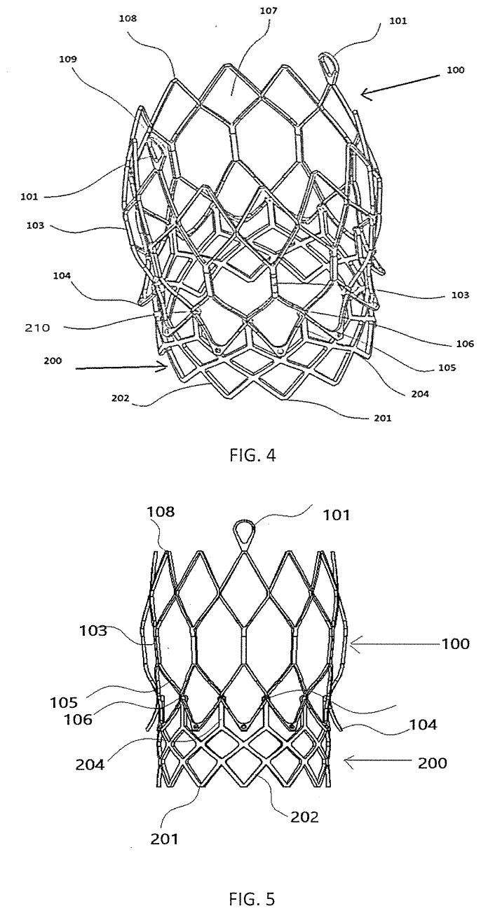

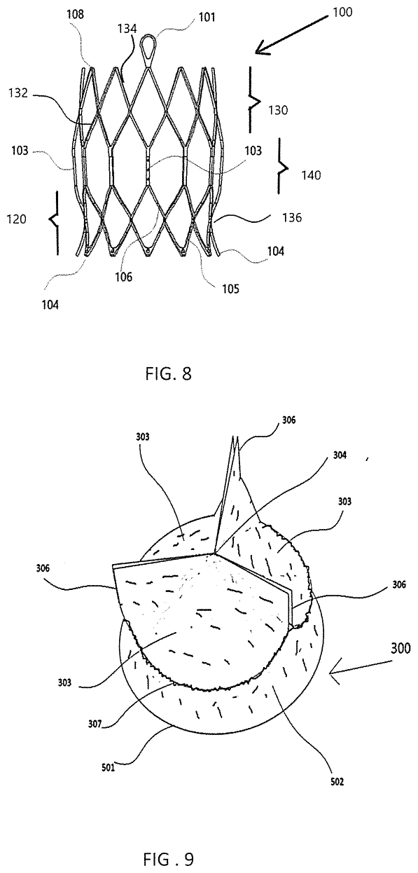

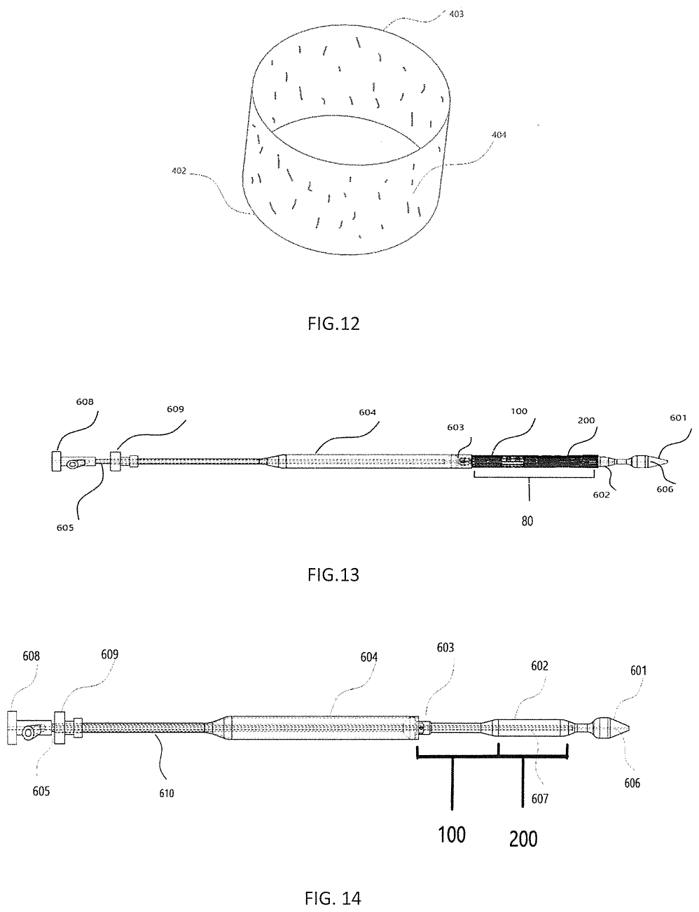

FIGS. 1-3 show the entire prosthesis 80. FIGS. 4 and 5 show the valve frame 100 and the annulus frame 200 and how they are interconnected. FIGS. 7 and 8 show the valve frame 100 alone, while FIG. 11 shows the annulus frame 200 alone. FIGS. 9 and 12 show the leaflet assembly 300 and the annulus skirt 400, respectively.

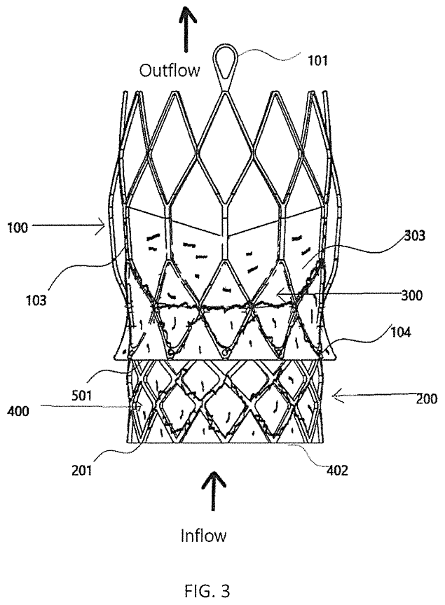

In a human heart, blood flows from the left ventricle through the aortic valve and towards the aorta. As used herein, the term “inflow side” shall mean the side of the prosthesis 80 from which blood from the left ventricle enters, and the term “outflow side” shall mean the side of the prosthesis 80 where blood exits and flows towards the aorta. These flow directions are shown by arrows “Inflow” and “Outflow” in FIG. 3.

Starting with FIGS. 4, 5, 7 and 8, the valve frame 100 is preferably made from a less rigid and more flexible material, such as a self-expandable material such as Nitinol™. The valve frame 100 has a generally cylindrical body having three rows of cells, with an inflow row 120 and an outflow row 130 separated by a central row 140. Each cell in the inflow row 120 of cells is made up of four struts (two first struts 105 and two second struts 106) that define a diamond shape, and with a plurality of spaced-apart inflow tips 104 defined by the vertices of two adjacent first struts 105. Each cell in the outflow row 130 of cells is made up of four struts (two third struts 132 and two fourth struts 134) that define a diamond shape, and with a plurality of spaced-apart outflow tips 108 defined by the vertices of two adjacent fourth struts 134. The vertices of two adjacent second struts 106 are connected to one end of a central beam 103, and the vertices of two adjacent third struts 132 are connected to the opposite end of central beam 103, such that each cell in the central row 140 are formed by two second struts 106, two central beams 103 and two third struts 132.

The inflow row 120 of cells is generally concave in a manner where the imaginary ring defined by the joints or connections 136 between the first struts 105 and the second struts 106 have the smallest diameter of any portion of the valve frame 100. Thus, the first struts 105 are generally curved inwardly from the tips 140 towards the connections 136, and then the second struts 106 are generally curved outwardly from the connections 136 towards the vertices that connect with the beam 103. The imaginary ring defined by the beams 103 define the largest diameter of any portion of the valve frame 100. The outflow row 130 is gently sloped from its largest diameter where the vertices of two adjacent third struts 132 are connected to the central beam 103, to the tips 108, where the imaginary ring defined by the tips 108 have the smallest diameter of any portion of the cells in the outflow row 130. As best shown in FIG. 3, the imaginary diameter defined by the connections 136 is smaller than the imaginary diameter defined by the tips 180.

At least one eyelet 101 is provided at a corresponding tip 108. The present embodiment shows the use of two eyelets 101. The eyelets 101 are used for securing the prosthesis 80 to a delivery system, as explained in greater detail below.

The leaflet assembly 300 is best shown in FIGS. 3, 6 and 9, and has three leaflets 303 having commissure edges 306 that are sewn to selected beams 103. The leaflets 303 define a center valve coaptation 304. Each leaflet 303 has another edge 307 that is stitched to an annular skirt 502, and the edges 307 define an annular connection edge that is stitched or otherwise connected to the connections 136 of the valve frame 100. The skirt 502 has an inflow edge 501 that is stitched or otherwise connected to the inflow tips 104 of the valve frame 100. Thus, the skirt 502 has a flared configuration similar to the flared configuration of the first struts 105. The skirt 502 and the leaflets 303 can be made from bovine pericardium or any other conventional tissue that has been treated prior to assembly using known tissue processing techniques (e.g., fixation, etc.). Three leaflets 303 are shown in use although it is also possible to have two leaflets.

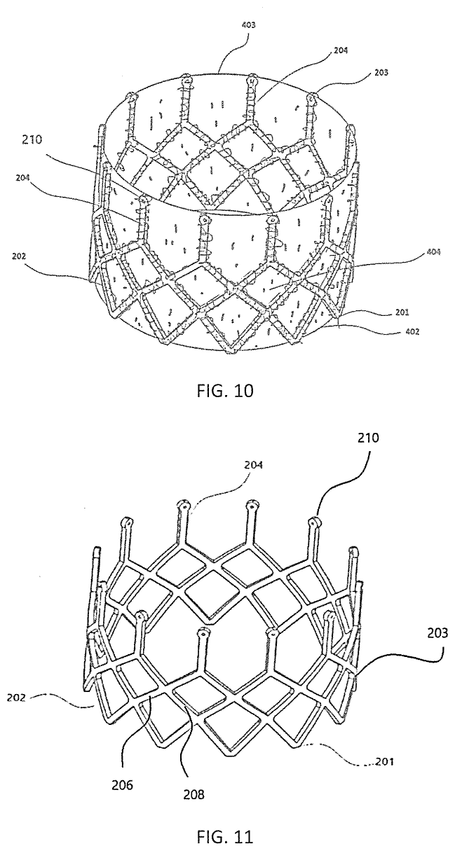

Referring now to FIGS. 10-11, the annulus frame 200 is preferably made from a more rigid material, such as cobalt chromium. The annulus frame 200 has two rows of cells, an inflow row 202 and an outflow row 203. Each cell in the two rows 202 and 203 is generally 4-sided or diamond-shaped, with the cells in each row 202 and 203 sharing a pair of struts 206 and 208. Inflow tips 201 are defined by vertices of the struts in the inflow row 202, and posts 204 extend from the outflow-facing vertices of the struts in the outflow row 203. Each post 204 has a rounded eyelet tip 210.

Referring now to FIGS. 10-12, the annulus skirt 400 is an annular band 404 of material which can be made from the same material as the leaflets 303 and the leaflet skirt 502. The inflow edge 402 of the band 404 is stitched or otherwise connected to the inflow tips 201, and the outflow edge 403 is stitched or otherwise connected to the eyelet tips 210. The profile or configuration for the annulus frame 200 and the skirt 400 is generally cylindrical with a generally constant diameter throughout, except that the imaginary ring defined by the inflow tips 201 can define a slight outward flared configuration, as best shown in FIG. 3.

The valve frame 100 and the leaflet assembly 300 is usually assembled separately from the annulus frame 200 and the annulus skirt 400. The combined valve frame 100 and leaflet assembly 300 is then connected to the combined annulus frame 200 and annulus skirt 400. Referring to FIGS. 1-5, the outflow end of the combined annulus frame 200 and annulus skirt 400 is inserted into the interior of the inflow end of the combined valve frame 100 and leaflet assembly 300, and more specifically, through the flared configuration defined by the ring of first struts 105. The eyelet tips 210 are adapted to engage at corresponding connections 136, which defines the smallest diameter so as to facilitate the engagement with the eyelet tips 210. This is best shown in FIG. 5. When fully assembled, the outflow end of the combined annulus frame 200 and annulus skirt 400 is received inside the inflow end of the combined valve frame 100 and leaflet assembly 300, as best shown in FIG. 3.

The valve frame 100 is preferably made by nickel titanium small tubing (e.g., 7 mm). The design can be created by laser-cutting the tubing and can be shape-set with the desired profile. At under 5 degrees Celsius, the frame becomes elastic and is easy to load onto the balloon 602 at a small size. When the temperature reaches 37 Celsius, the frame returns to its shape-set profile.

The annulus frame 200 can be made by cobalt-chrome small tubing (e.g., 7 mm). The design can be laser-cut on the tubing and expanded like a shaped cylinder with a 23 mm, 26 mm, 29 mm, or 32 mm diameter.

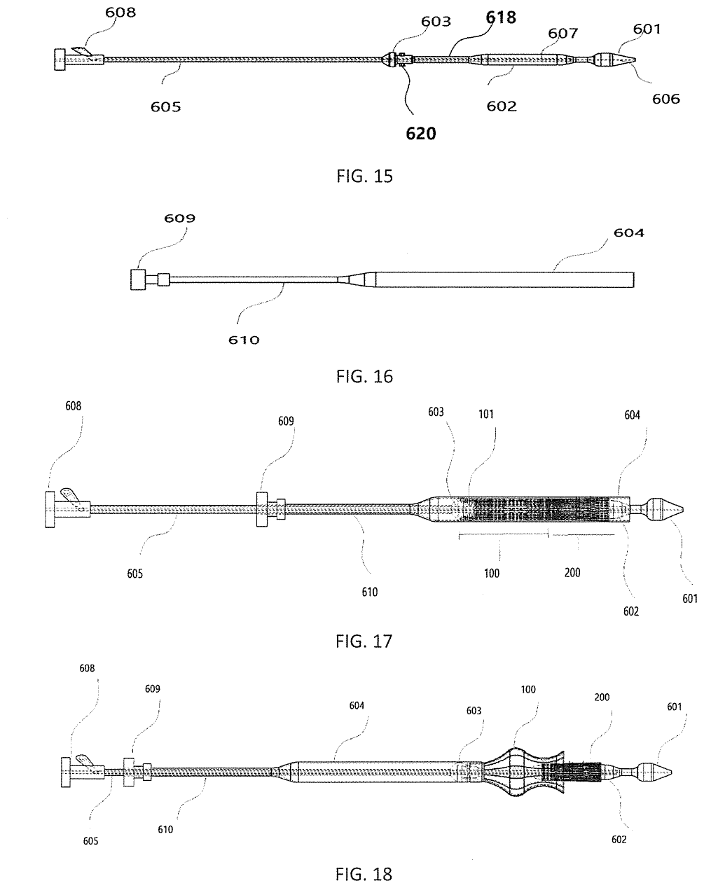

FIGS. 13-17 illustrate a delivery system 600 that is adapted for use in delivering the prosthesis 80 to an aortic annulus, and deploying it at the aortic annulus. The delivery system 600 includes a balloon catheter (shown in FIG. 15) and a sheath assembly (shown in FIG. 16) that is sized and configured to ensheath and release the prosthesis 80.

The balloon catheter has a shaft 605 that extends from a T-junction 608 to a tapered distal tip 601. An inflatable balloon 602 is provided on the shaft 605 adjacent the tapered distal tip 601. A valve frame seat 618 is provided immediately proximal to the balloon 602, which is essentially a portion of the shaft 605. A guidewire tube 607 extends from the T-junction 608 through a lumen of the shaft 605 and has a portion 606 that extends through the distal tip 601. An ear hub 603 is provided on the shaft 605 immediately proximal to the valve frame seat 618. The ear hub 603 has one or more notches 620 which is adapted to be received by the opening in a corresponding eyelet 101. The remainder of the catheter can be embodied using principles and catheters that are well-known in the art, and will not be described as this is well-known to a person skilled in the art.

The sheath assembly has a hollow shaft 610 with a lumen that is sized to receive the shaft 605 of the balloon catheter. A blocking handle 609 is provided at the proximal end of the hollow shaft 610 and functions to move the capsule 604 distally (to ensheath the prosthesis 80), and to withdraw the capsule 604 proximally (to release the prosthesis 80). The T-junction 608 can act as a block to limit the proximal travel of the blocking handle 609. A capsule 604 is provided on the distal end of the hollow shaft 610. As shown in FIGS. 13, 14 and 17, the blocking handle 609 can extend from adjacent the T-junction 608 to a position along the shaft 605 that is closer to the balloon 602. The shaft 605 is received inside the lumen of the hollow shaft 610, and the capsule 604 is adapted to cover the position of the balloon 602 and to be withdrawn proximally to completely expose the balloon 602 and the valve frame seat 618. As shown in FIGS. 13 and 17, the prosthesis 80 is positioned at the location of the balloon 602 and the valve frame seat 618. Specifically, the cobalt chromium annulus frame 200 is crimped on to the balloon 602, and the valve frame 100 is crimped on to the location of the valve frame seat 618. The sheath assembly is then advanced distally so that the capsule 604 completely covers both the valve frame 100 and the annulus frame 200.

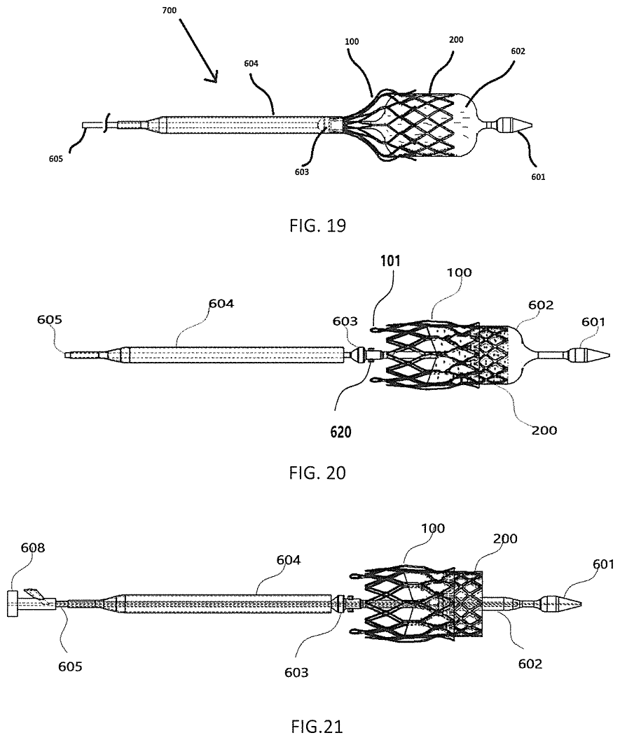

When the prosthesis 80 has been delivered to the location of a native aortic annulus inside a patient, the sheath assembly can be withdrawn so that the capsule 604 is withdrawn such that the distal end of the capsule 604 is at about the location of the ear hub 603 (see FIG. 18), and in this position, the valve frame 100 begins to self-expand while the annulus frame 200 remains crimped. Next, the balloon 602 is inflated (FIG. 19) to expand the annulus frame 200. The sheath assembly is then further withdrawn (FIG. 20) so that the capsule 604 releases the ear hub 603 and the eyelets 101 that are secured to the notches 620. Self-expansion of the eyelets 101 will cause the eyelets 101 to disengage from the notches 620. The balloon 602 is then deflated (FIG. 21) so that the balloon catheter can also be withdrawn.

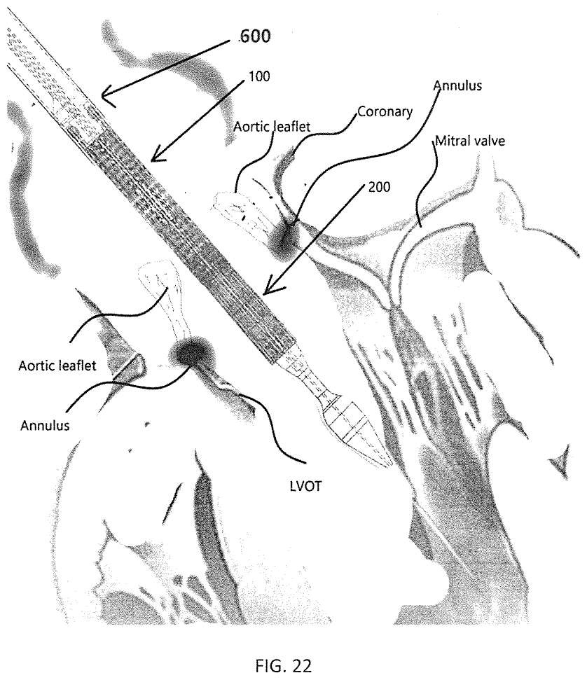

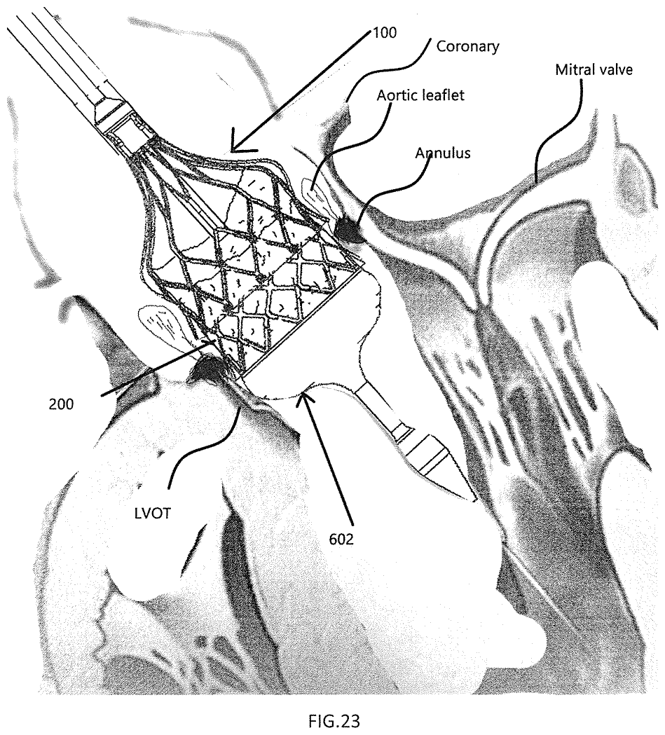

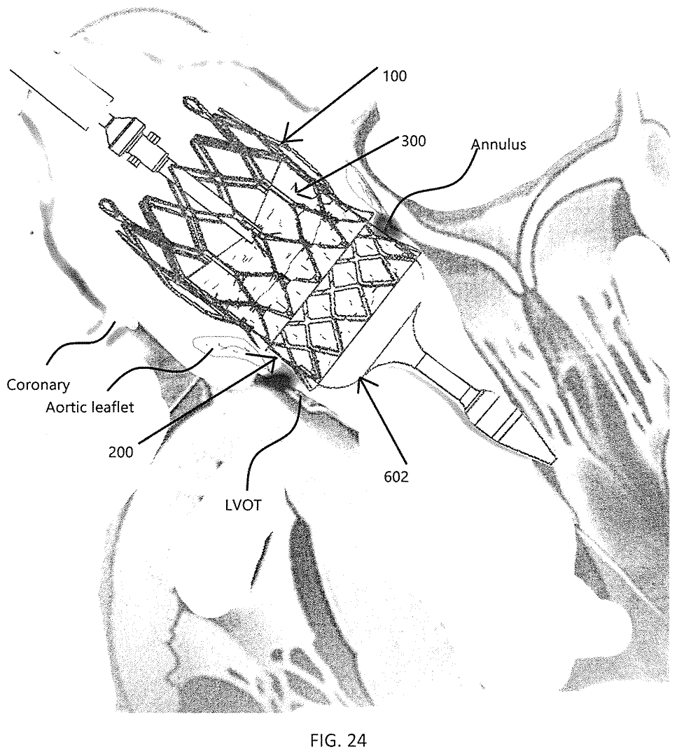

FIGS. 22-25 illustrate how the delivery system 600 delivers and deploys the prosthesis 80 at the aortic annulus of a human heart. Starting with FIG. 22, the delivery system 600 with the prosthesis 80 ensheathed by the capsule 604 is delivered from the aorta through the aortic annulus using transcatheter techniques that are well-known in the art. As shown in FIG. 22, the balloon catheter is preferably positioned so that the annulus frame 200 is at the location of the aortic annulus. Next, the capsule 604 is withdrawn such that the distal end of the capsule 604 is at about the location of the ear hub 603 (see FIGS. 18 and 23). In this position, the valve frame 100 begins to self-expand while the annulus frame 200 remains crimped, and then the balloon 602 is inflated (see FIGS. 19 and 23) to expand the annulus frame 200. The sheath assembly is then further withdrawn (see FIGS. 20 and 24) so that the capsule 604 releases the ear hub 603 and the eyelets 101 that are secured to the notches 620. The balloon 602 is then deflated so that the balloon catheter can also be withdrawn.

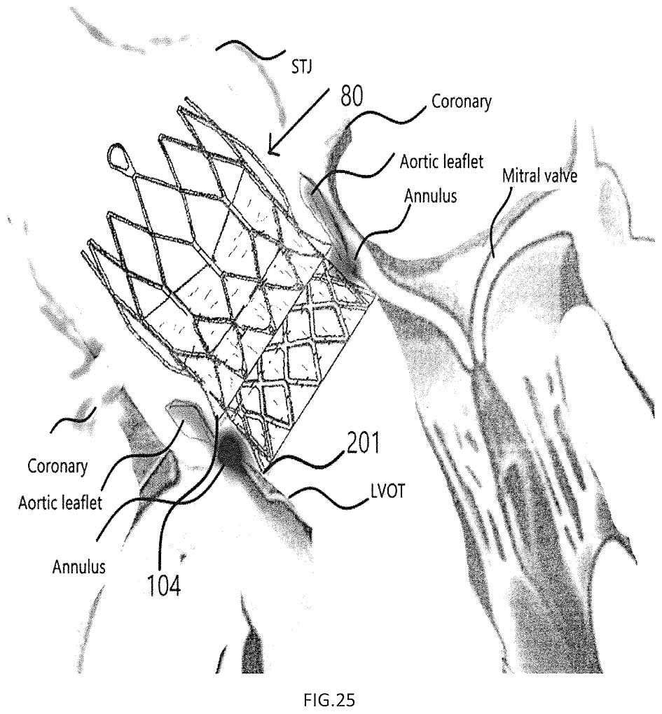

FIG. 25 shows the prosthesis 80 implanted at the aortic annulus. The annulus frame 200 is secured at the aortic annulus and the flared configuration defined by the tips 104 in the ring of first struts 105 of the inflow row 120 of cells for the valve frame 100 is positioned below the native aortic leaflets to provide an effective securing mechanism to prevent the prosthesis 80 from moving towards the inflow side. Similarly, the slight flare defined by the ring of inflow tips 201 prevents the prosthesis 80 from moving towards the outflow side.

The present invention provides a number of unique features and benefits.

First, the annulus frame 200 is expanded by a balloon with a strong frame radial force, so it can be used for patients with AS (aortic stenosis) at the location of calcification in the aortic annulus and the LVOT.

Second, when the balloon expands the annulus frame 200, the inflow tips 201 are flared to prevent that the prosthesis 80 from moving towards the outflow side, so that the prosthesis can be used for patients with AI (aortic insufficiency) and AR (aortic regurgitation).

Third, since the valve frame 100 is made of Nitinol™, the frame is easy to shape with the preferred profile to have a flare that can be used to anchor at the aortic annulus, so that the prosthesis is suitable for use with patients suffering from Al (aortic insufficiency) and AR (aortic regurgitation).

Fourth, the prosthesis 80 allows the procedure to be very precise. Based on the two different materials for the Nitinol™ self-expanding valve frame 100 and the cobalt chromium annulus frame 200, when the prosthesis 80 is partially exposed by the capsule 604, the valve frame 100 slowly self-expands while the annulus frame 200 is still crimped on the balloon 602, thereby avoiding a “jump” or sudden expansion by the annulus frame 200. This allows the physician time to position the prosthesis 80 and to inflate the balloon 602.

While the description above refers to particular embodiments of the present invention, it will be understood that many modifications may be made without departing from the spirit thereof. The accompanying claims are intended to cover such modifications as would fall within the true scope and spirit of the present invention.

What is claimed is:

1. A heart valve assembly, comprising:
a valve frame having a leaflet assembly sutured to the valve frame, the leaflet assembly having a plurality of leaflets;
an annulus frame having an annulus skirt sutured to the annulus frame; and
wherein the annulus frame is connected to the valve frame, and the valve frame and the annulus frame are made from different materials.

2. The assembly of claim 1, wherein the valve frame is made from a self-expanding material and the annulus frame is made from a balloon expandable material.

3. The assembly of claim 1, wherein the annulus frame is made from cobalt-chromium.

4. The assembly of claim 3, wherein the valve frame is made from Nitinol.

5. The assembly of claim 1, wherein the assembly has an inflow side, and an outflow side, and the valve frame has a flared inflow end that is configured to be positioned below the native aortic leaflets to provide an effective securing mechanism to prevent the assembly from moving towards the inflow side.

6. The assembly of claim 5, wherein the annulus frame has an inflow side that is flared to prevent that the annulus frame from moving towards the outflow side of the assembly.

7. The assembly of claim 1, wherein the valve frame has a flared inflow end and the annulus frame has an outflow end having a plurality of posts, with each of the plurality of posts having an outflow tip that is adapted to engage a connection location on the flared inflow end of the valve frame.

8. The assembly of claim 7, wherein the plurality of posts extends inside the valve frame.

9. The assembly of claim 8, wherein the flared inflow end of the valve frame has a concave configuration, and the connection locations are positioned at a base of the concavity, with the connection locations defining a ring that has the smallest diameter of the valve frame.

10. A system, comprising:
a heart valve assembly comprising:
a valve frame having a leaflet assembly sutured to the valve frame, the leaflet assembly having a plurality of leaflets;
an annulus frame having an annulus skirt sutured to the annulus frame;
wherein the annulus frame is connected to the valve frame, and the valve frame and the annulus frame are made from different materials;
a delivery system having:
a balloon catheter having a shaft having a distal end, a balloon provided on the shaft adjacent the distal end, and a valve frame seat located on the shaft directly proximal to the balloon;
a sheath assembly having a capsule that slidably covers the balloon and the valve frame seat; and
wherein the valve frame is seated around the valve frame seat and the annulus frame is seated over the balloon.

11. The system of claim 10, wherein:
an eyelet provided on the valve frame;
an ear hub provided on the shaft immediately proximal to the valve frame seat; and
the eyelet engages the ear hub when the valve frame is seated around the valve frame seat and the annulus frame is seated over the balloon.

12. The system of claim 10, wherein the valve frame is made from a self-expanding material and the annulus frame is made from a balloon expandable material.

13. The system of claim 10, wherein the annulus frame is made from cobalt-chromium.

14. The system of claim 13, wherein the valve frame is made from Nitinol.

15. A method of delivering a heart valve assembly to an aortic annulus which has native aortic leaflets, comprising:
providing a heart valve assembly comprising:
a valve frame having a leaflet assembly sutured to the valve frame, the leaflet assembly having a plurality of leaflets;
an annulus frame having an annulus skirt sutured to the annulus frame;
wherein the annulus frame is connected to the valve frame, and the valve frame and the annulus frame are made from different materials;
providing a delivery system having:
a balloon catheter having a shaft having a distal end, a balloon provided on the shaft adjacent the distal end, and a valve frame seat located on the shaft directly proximal to the balloon;
a sheath assembly having a capsule; and
crimping the heart valve assembly on to the balloon catheter by seating the valve frame around the valve frame seat and the annulus frame over the balloon;
sliding the capsule to cover the balloon and the valve frame seat;
advancing the delivery system with the heart valve assembly ensheathed by the capsule to the location of an aortic annulus in a human heart with the annulus frame at the location of the aortic annulus;
withdrawing the capsule such that the capsule does not cover the valve frame, and such that the valve frame begins to self-expand while the annulus frame remains crimped;
inflating the balloon to expand the annulus frame;
deflating the balloon; and
withdrawing the balloon catheter.

16. The method of claim 15, further including:
providing an eyelet on the valve frame;
providing an ear hub on the shaft immediately proximal to the valve frame seat; and
wherein the eyelet engages the eyelet when the valve frame is seated around the valve frame seat and the annulus frame is seated over the balloon.

17. The method of claim 15, further including providing the valve frame as a self-expanding material and the annulus frame as a balloon expandable material.

18. The method of claim 15, wherein the annulus frame is made from cobalt-chromium.

19. The method of claim 18, wherein the valve frame is made from Nitinol.
